(19) United States
(12) Patent Application Publication
(10) Pub. No.: US 2022/0387767 A1
(43) Pub. Date: Dec. 8, 2022

(54) Hourglass Balloon for Transcatheter Aortic Valve Implantation

(71) Applicant: Istanbul Medipol Universitesi, lstanbul (TR)

(72) Inventor: Yahya YILDIZ, Istanbul (TR)

(21) Appl. No.: 17/775,814

(22) PCT Filed: Nov. 26, 2020
(86) PCT No.: PCT/TR2020/051171

§ 371 Date: May 10, 2022

(30) Foreign Application Priority Data
Nov. 26, 2019 (TR) TR 2019/18438
Nov. 26, 2019 (TR) TR 2019/18439

(51) Int. Cl. A61M 25/10

(52) U.S. Cl. CPC A61M 25/1002; A61M 25/1011; A61M 2025/1068; A61M 2025/1079; A61M 2025/1097; A61M 2210/127

(57) ABSTRACT

An hourglass shaped three-chambered and tunneled balloon catheter suitable for use in the placement of an aortic valve during the transcatheter aortic valve implantation (TAVI) process.

### TECHNICAL FIELD

The present invention is related to an hourglass shaped, 3 (three) chambered balloon catheter, which is suitable for use in the placement of the aortic valve in a transcatheter aortic valve implantation (TAVI) process, and which optionally includes a tunnel.

### PRIOR ART

The aortic valve is usually a valve that opens while the blood is pumped from the heart to the body; it works one-way, it closes due to the pressure decrease after the blood is discharged from the heart to the aorta, that is, to the large vessels. It prevents the discharged blood from returning to the heart, in other words, it is a one-way valve. There are conditions or diseases caused by aortic valve stenosis or insufficiency or a combination of both, or conditions caused by the aortic valve not opening and closing properly. This situation puts extra pressure or strain on the heart and can cause shortness of breath, swollen ankles, chest pain, dizziness, and sometimes fainting.

Normal treatment for people with aortic valve problems is open heart valve surgery. However, open heart surgery can be considered too risky for people who are very ill or have many other medical problems.

It has been shown that surgical aortic valve replacement (replacement) can increase morbidity (disease duration) and mortality in some cases, and Transcatheter aortic valve implantation (TAVI) process can be beneficial. The process of inserting aortic valve without removing the old valve by entering the inguinal artery (femoral artery) without using stitches is called TAVI.

(TAVI) is a procedure that allows an aortic valve to be implanted using a long and narrow tube called a catheter or a carrier. Usually, the catheter is inserted through a large blood vessel in the groin or through a small incision in the chest.

This new technique allows the surgeon to reach the heart without opening the chest. The benefit or reason for preference of such a TAVI procedure is that it is considered to be beneficial in patients suitable for TAVI due to the high risk of complications from open heart surgery for aortic valve replacement.

Transcatheter aortic valves are specially designed for this procedure. The valve is made from the heart of a cow or a pig, in other words from natural tissue. The natural tissue is redesigned, attached to a flexible and expanding or expandable mesh framework. To insert it into the heart, the valve is compressed around or inside a catheter. Then the catheter comprising the aortic valve is brought over the aortic valve and placed on the existing valve. The catheter placed on the existing valve is directed to the aortic valve opening in the heart or where the valve is located. After the new valve is inserted, the catheter is removed. It starts working immediately after the new valve is placed.

Several tests such as electrocardiogram, echocardiogram, computed tomography (CT) scan, and angiogram are required to determine the benefit of TAVI valve application.

The TAVI process consists of 3 basic steps;
1. Pre-inflation or semi-expansion (dilatation) by means of a balloon in order to expand the narrow (stenotic) valve just before the valve is placed,
2. Placement of the TAVI valve over the aortic valve
3. Placing a balloon inside the TAVI valve placed in the aortic valve position and inflating, placing the valve or holding the stented valve structure by the surrounding tissues and ensuring that the stented structure expands and takes its final shape. The valve used here can be self-expanding or non-self-expanding, that is, expanded with a balloon, shaped or attempted to be placed in its appropriate position.

During said process, an amount of opening of the valve is provided without full inflation in the step 1, for example by half inflation. During this inflation process, the blood pressure and pulse rate (hemodynamics) of the patient may be slightly impaired. Also, a particle can detach from the calcified valve during the inflation process and can pass to different organs, such as the brain, causing damage, for example paralysis.

In addition, the heart conduction system is located on the outer side of the inflated, in other words, expanded valve, and this conduction system may be damaged due to the pressure exerted by the inflated balloon. Damage to this conduction system will lead to conduction problems, causing permanent damage to the patient's heart, thereby necessitating the need for a pacemaker to be inserted.

Similar problems also occur during the inflation process performed in step 3. Here, by inflating the balloon and applying pressure to the valve outward, the calcareous structures in the surrounding tissue may be ruptured, and the need for permanent a pacemaker implantation may arise with the damage of the heart's conduction system in this part.

The inventors aim to develop balloon catheters that will ensure the effective placement of the TAVI valve and allow this procedure to be performed without applying excessive pressure to calcareous structures in the surrounding tissue or applying it as desired or needed, and without damaging the conduction system of the heart.

### BRIEF DESCRIPTION OF THE INVENTION

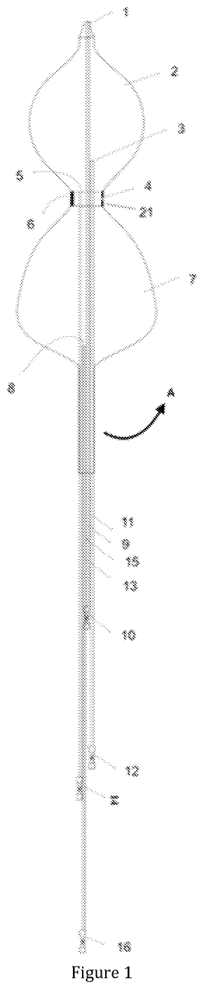

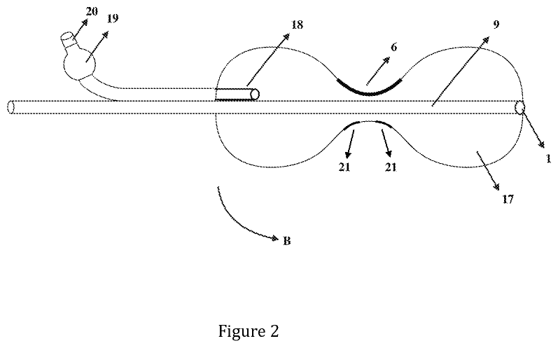

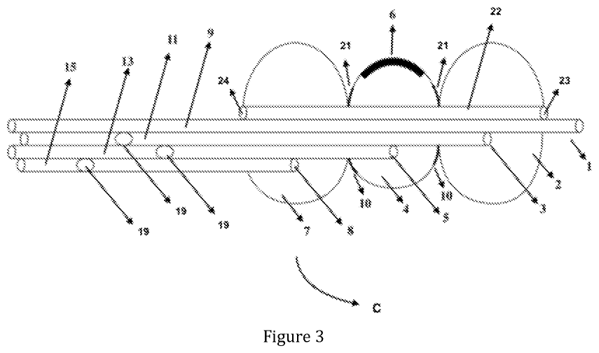

The present invention relates to an hourglass shaped triple balloon catheter (A and B and C) suitable for use in aortic valve placement during a transcatheter aortic valve implantation (TAVI) procedure (FIG. 1, FIG. 2 and FIG. 3). The catheter according to the invention can be in the form of a multi-segment and multi-lumen (FIG. 1) or single-segment and single-lumen (FIG. 2), or optionally it may comprise a tunnel (17) that passes through the balloon (FIG. 3). The feature of the balloon catheter in hourglass form according to the invention is that it contains a non-elastic, rigid part (6) in the part located in the middle of the hourglass form. In this way, it is possible to use an hourglass balloon catheter with both forms (single lumen and multi-lumen) without damaging the conduction system of the heart. In addition, if it includes the tunnel (17) optionally, it is possible to provide blood flow from the heart to the brain during the operation and to keep the patient's blood pressure balanced.

The multi-lumen balloon catheter (A) according to the invention includes the left ventricular outflow balloon (2) (LVOT) which enables the is part of the TAVI valve to be opened and fixed to the sub-aortic intraventricular section when inflated, the aortic valve dilatation balloon (4) that opens or expands the aortic valve structures when inflated, and the ascending aortic dilatation balloon (7) parts that allow the 3rd part of the valve to be placed in the aorta that comes out over the coronary sinus by opening or expanding the third part of the TAVI valve when inflated; characterized in that it includes the lumen leading to the proximal end (9), the left ventricular outflow balloon lumen (11), the aortic valve dilatation balloon lumen (13) and the ascending aortic dilatation balloon lumen (15) and it includes a rigid (non-elastic) part (6) on the aortic valve dilatation balloon (4) (in the middle of the hourglass).

The single-lumen balloon catheter (B) according to the invention includes an hourglass shaped balloon (17) that allows all parts of the TAVI valve to be opened and placed when inflated, characterized in that it includes the lumen (9) leading to the proximal end that allows the guide wire to pass through it, the hourglass balloon lumen (18) that allows the hourglass balloon to be inflated, hourglass pilot balloon (19), and a one-way valve (20) and in that a rigid (non-elastic) part (6) is present in the middle part of the hourglass.

The tunnel balloon catheter (C) according to the preferred embodiment of the invention includes the left ventricular outflow balloon (2) which enables the 1st part of the TAVI valve to be opened and fixed to the sub-aortic intraventricular (LVOT) part when inflated, the aortic valve dilatation balloon (4) that opens or expands the aortic valve structures when inflated, the ascending aortic dilatation balloon (7) parts that allow the third part of the valve to be placed in the aorta that comes out over the coronary sinus by opening or expanding the third part of the TAVI valve when inflated; characterized in that it includes the lumen leading to the proximal end (9), the left ventricular outflow balloon lumen (11), the aortic valve dilatation balloon lumen (13) and the ascending aortic dilatation balloon lumen (15) a rigid (non-elastic) part (6) on the aortic valve dilatation balloon (4) (in the middle of the hourglass) and a tunnel (17) passing through hourglass balloon structure.

The invention aims to eliminate the problems caused by the balloons used in the prior art by means of the multi-lumen balloon catheter (A), single lumen balloon catheter (B) and tunnel balloon catheter (C). Balloon catheters (A, B and C) according to the invention can be used in the placement of all types of TAVI valves, and thanks to the non-elastic part (6) on the aortic valve dilatation balloon, by inflating the balloon during use more force is applied to the fibrous structures and less force is applied to the soft tissue between the fibrous structures. Thus, the possibility of damage to the vascular structures behind the aortic cusp and especially the conduction paths is reduced or prevented. Therefore, the possibility of a situation requiring the implantation of a permanent pacemaker is reduced.

### BRIEF DESCRIPTION OF THE FIGURES

In FIG. 1 and FIG. 2, representative drawings of the multi-lumen balloon catheter (A) and single lumen balloon catheter (B) according to the invention are shown.

The reference numbers and corresponding parts in the figures are listed below;

1: Proximal End

2: Left Ventricular Outflow Balloon

3: Left Ventricular Outflow Balloon Inflation End

4: Aortic Valve Dilatation Balloon

5: Aortic Valve Dilatation Balloon Inflation End

6: Rigid Part

7: Ascending Aortic Dilatation (dilating the aortic valve position and insertion) Balloon

8: Ascending Aortic Dilatation Balloon Inflation End

9: Lumen leading to the proximal end number 1

10: Valve on lumen number 9

11: Left Ventricular Outflow Balloon Lumen

12: Valve on lumen number 11

13: Aortic Valve Dilatation Balloon Lumen

14: Valve on lumen number 13

15: Ascending Aortic Dilatation Balloon Lumen

16: Valve on lumen number 15

17: Hourglass balloon

18: Hourglass balloon lumen

19: Pilot balloon

20: One-way valve

21: Radio-opaque strip

22: Tunnel

23: Tunnel proximal end

24: Tunnel distal end

### DETAILED DESCRIPTION OF THE INVENTION

The multi-lumen balloon catheter (A), single lumen balloon catheter (B) and tunnel balloon catheter (C) according to the invention take the form of an hourglass when inflated (FIG. 1, FIG. 2 and FIG. 3).

Lumens contained in the multi-lumen balloon catheter (A) and tunnel balloon catheter (C) according to the invention enables each part of the balloon catheter in hourglass form to be inflated and discharged independently from each other. This gives the user more control over the use of the balloon during the operation.

In the single lumen balloon catheter (B) according to the invention, the balloon in hourglass form is inflated and discharged through a single lumen. This situation provides convenience to the user.

The term “hourglass form” used here refers to a structure with three compartments in total, where the two parts constituting the said structure are connected to each other with a narrower part compared to the others. In other words, the hourglass form refers to the structures with two wide parts and a relatively narrow part connecting the two wide parts there-between. Said parts may be separated from each other by sections and/or curtains, or there may not be any sections between these parts.

The terms “rigid part” and “non-elastic part” used within the scope of the invention are equivalent to each other and can be used interchangeably. The terms “rigid part” and “non-elastic part” refer to all structures that have lower elasticity, higher strength, and do not change shape when the balloon is filled with air compared to the material forming the other parts of the hourglass balloon.

In the hourglass shaped balloon catheter (A, B or C) according to the invention, the rigid part (6) may cover half or less (for example, 10%-45% of the surface area) or more (55%-90% of the surface area) or all of the surface of the aortic valve dilatation balloon in the multi-lumen catheter (A) or in the single-lumen catheter (B) or the tunnel balloon catheter (C) it may cover half or less of the surface of the hourglass part or it may cover all of it. In a preferred embodiment of the invention, the rigid part (6) is made of a radio-opaque material. Thereby the practitioners can determine that this rigid part (6) on the catheter is placed in the right place during the procedure, and then the procedure can be continued by inflating the balloon without damaging the heart conduction systems in the surrounding tissue.

In a preferred embodiment of the invention, in the multi-lumen balloon catheter (A) and the tunnel balloon catheter (C) according to the invention, the lumen (9) leading to the proximal end number 1, the left ventricular outflow balloon lumen (11), the aortic valve dilatation balloon lumen (13), and the ascending aortic dilatation balloon lumen (15) are optionally provided with a valve, valves 10, 12, 14, 16, respectively that are used to provide and control fluid flow.

In a preferred embodiment of the invention, in the multi-lumen balloon catheter (A) and the tunnel balloon catheter (C), the left ventricular outflow balloon lumen (11) extends into the left ventricular outflow balloon (2), and the balloon is inflated when desired by left ventricular outflow balloon inflation end (3) located on said lumen. Air transmission through said lumen (11) to the balloon (2), through the inflation end (3) is controlled by a valve (12) located on the lumen.

In a preferred embodiment of the invention, in the multi-lumen balloon catheter (A) and the tunnel balloon catheter (C), the aortic valve dilatation balloon lumen (13) extends into the aortic valve dilatation balloon (4), wherein the balloon is inflated when desired by the aortic valve dilatation balloon inflation end (5) located on the said lumen. Fluid transmission through said lumen (13) to the balloon (4) via the inflation end (5) is controlled by a valve (14) located on the lumen.

In a preferred embodiment of the invention, in the multi-lumen balloon catheter (A) and the tunnel balloon catheter (C), the ascending aortic dilatation balloon lumen (15) extends into the ascending aortic dilatation balloon (7), and wherein the balloon is inflated when desired by aortic valve dilatation balloon inflation end (8) located on said lumen. Fluid transmission through said lumen (15) to the balloon (7) via the inflation end (8) is controlled by a valve (16) located on the lumen.

In one aspect, the invention relates to a multi-lumen hourglass shaped balloon catheter (A) and optionally a version comprising a tunnel, and a tunneled hourglass shaped balloon catheter (C), and it comprises the left ventricular outflow balloon (2) which enables the 1st part of the TAVI valve to be opened and fixed to the sub-aortic intraventricular (LVOT) part when inflated, the aortic valve dilatation balloon (4) that opens or expands the aortic valve structures when inflated, the ascending aortic dilatation balloon (7) parts that allow the 3rd part of the valve to be placed in the aorta that extends out over the coronary sinus by opening or expanding the 3rd part of the TAVI valve when inflated; characterized in that it further comprises the lumen leading to the proximal end (9), the left ventricular outflow balloon lumen (11), the aortic valve dilatation balloon lumen (13), the ascending aortic dilatation balloon lumen (15) and a rigid (non-elastic) part (6) located on the aortic valve dilatation balloon (4).

In another aspect, the invention is a single-lumen hourglass shaped balloon catheter (B), it includes an hourglass balloon (17) that allows the 1st part of the TAVI valve to be opened and fixed to the sub-aortic valve intraventricular (LVOT) part when inflated, and that also allows the aortic valve structures to be opened and expanded and 3rd part of the valve to be placed in the aorta that comes out over the coronary sinus by opening and expanding 3rd part of the TAVI valve; characterized in that it comprises the lumen (9) leading to the proximal end, an hourglass balloon lumen (18) and a rigid (non-elastic) part (6) in the middle part of the hourglass.

The term “middle part of the hourglass” used in the text refers to the narrow part connecting the two wide parts in an hourglass shaped structure, and it is equivalent to the phrase “Aortic Valve Dilatation Balloon (4)” in a multi-lumen catheter and both expressions can be used interchangeably.

The multi-lumen balloon catheter (A), single lumen balloon catheter (B) and tunnel balloon catheter (C) according to the invention can be made of any material suitable for the physiological environment and known to be usable under surgical or angiographic conditions.

In a preferred embodiment of the invention, the proximal end (1) in the multi-lumen balloon catheter (A) or the tunnel balloon catheter (C), can have a marker of any length and shape, for example strip, made of radiopaque material in at least one of the lumen leading to the proximal end number 1 (9), the left ventricular outflow balloon lumen (11), the left ventricular outflow balloon (2), aortic valve dilatation balloon lumen (13), aortic valve dilatation balloon (4), ascending aortic dilatation balloon lumen (15), and aortic valve dilatation balloon (7) components.

In a particularly preferred embodiment of the invention, the tunnel balloon catheter (C) has a radio opaque strip or strips (10) on the aortic valve dilatation balloon (4), in the region of this balloon close to the left ventricular outflow balloon (2) and the ascending aortic dilatation balloon (7).

In a preferred embodiment of the invention, in the multi-lumen balloon catheter (A) and the tunnel balloon catheter (C), optionally a pilot balloon (19) is available on the left ventricular outflow balloon lumen (11), the aortic valve dilatation balloon lumen (13), and the ascending aortic dilatation balloon lumen (15).

Pilot balloons (19) on the multi-lumen balloon catheter (A), single-lumen balloon catheter (B) and tunnel balloon catheter (C) are components which provide monitoring of the pressure of different parts of the hourglass balloon (A, B, C) which is in an invisible point inside the patient's body.

In an embodiment of the present invention, a one-way valve (20) is provided at any point between the proximal end (23) and the distal end (24) of the tunnel (22) in the single lumen balloon catheter (B) and the tunnel balloon catheter (C). Said valve may be in the form of two valves or three valves. In a preferred embodiment of the invention, said one-way valve is located in the middle of the tunnel (22) or at a point close to the distal end (24) or near the proximal end.

The one-way valve (20) located in the single lumen balloon catheter (B) prevents the pressure inside the balloon from releasing again after the balloon is inflated.

In an embodiment of the present invention, in the single lumen balloon catheter (B) and the tunnel balloon catheter (C), the lumen (9) leading to the proximal end creates a connection between the external environment and the intravascular part. The catheter is placed in the desired location thanks to a guide wire passed through the lumen (9) leading to the proximal end.

The hourglass balloon (17) in the single lumen balloon catheter (B) consists of a single piece in the form of an hourglass or 3 connected parts and can be inflated from a single point.

In a preferred embodiment of the invention, the marker, for example the strip having any length and shape made of radio-opaque material, may be present in at least one of the components such as the proximal end (1) located in the single lumen balloon catheter (B), the Hourglass balloon (17), the Hourglass balloon pilot balloon (19), and the lumen (9) leading to the proximal end number 1.

The embodiments of the invention can be combined when technically applicable.

The embodiments were described here to include certain features/elements.

The disclosure also covers other implementations that essentially contain or consist of said features/elements.

The applications that have been specifically and explicitly described herein may serve as a basis for disclaimer, alone or in combination with one or more other applications.

1. A balloon catheter in hourglass form comprising a lumen leading to a proximal end and a rigid (non-elastic) part in the middle of the hourglass wherein the rigid (non-elastic) part covers 10%-45% of the surface area of an aortic valve dilatation balloon.

2. A balloon catheter according to claim 1, wherein it is a multi lumen (A), single lumen (B) or tunnel catheter (C).

3. A balloon catheter in hourglass form according to claim 1, wherein the rigid part is made of a radio-opaque material.

4. A multi lumen balloon catheter in hourglass form according to claim 2, comprising a left ventricular outflow balloon which enables the first part of the TAVI valve to be opened and fixed to the sub-aortic intraventricular (LVOT) part when inflated, the aortic valve dilatation balloon that opens or expands the aortic valve structures when inflated, the ascending aortic dilatation balloon parts that allow the 3rd part of the valve to be placed in the aorta that extends out over the coronary sinus by opening or expanding the 3rd part of the TAVI valve when inflated; and the lumen leading to the proximal end, a left ventricular outflow balloon lumen, an aortic valve dilatation balloon lumen and an ascending aortic dilatation balloon lumen.

5. A tunnel balloon catheter in hourglass form according to claim 2, comprising a left ventricular outflow balloon which enables the 1st part of the TAVI valve to be opened and fixed to the sub-aortic intraventricular (LVOT) part when inflated, an aortic valve dilatation balloon that opens or expands the aortic valve structures when inflated, an ascending aortic dilatation balloon parts that allow the 3rd part of the valve to be placed in the aorta that comes out over the coronary sinus by opening or expanding the 3rd part of the TAVI valve when inflated, and the lumen leading to proximal end parts.

6. A multi lumen balloon catheter or tunnel hourglass shaped balloon catheter according to claim 2, wherein a left ventricular outflow balloon lumen, an aortic valve dilatation balloon lumen and an ascending aortic dilatation balloon lumen on the balloon allow each part of the balloon catheter to be inflated and discharged independently from each other.

7. A multi-lumen balloon catheter and a tunnel hourglass shaped balloon catheter according to claim 6, wherein the lumen leading to the proximal end number 1, a left ventricular outflow balloon lumen, an aortic valve dilatation balloon lumen, and an ascending aortic dilatation balloon lumen are provided with a valve that is used to provide and control air flow and optionally fluid flow.

8. A multi-lumen balloon catheter and a tunnel hourglass shaped balloon catheter according to claim 2, wherein a left ventricular outflow balloon lumen extends into a left ventricular outflow balloon, and wherein the balloon is inflated when desired by a left ventricular outflow balloon inflation end located on the said lumen.

9. A multi-lumen balloon catheter and a tunnel hourglass shaped balloon catheter according to claim 8, wherein the air transmission through the inflation end to the balloon via the left ventricular outflow balloon lumen is controlled by a valve.

10. A multi-lumen balloon catheter and a tunnel hourglass shaped balloon catheter according to claim 2, wherein an aortic valve dilatation balloon lumen extends into an aortic valve dilatation balloon, and wherein the balloon is inflated when desired by an aortic valve dilatation balloon inflation end located on said lumen.

11. A multi-lumen balloon catheter and a tunnel hourglass shaped balloon catheter according to claim 10, wherein the air transmission through inflation end to the balloon via the aortic valve dilation balloon lumen is controlled by a valve.

12. A multi-lumen balloon catheter and a tunnel hourglass shaped balloon catheter according to claim 2, wherein an ascending aortic dilatation balloon lumen extends into an ascending aortic dilatation balloon, and wherein the balloon is inflated when desired by an aortic valve dilatation balloon inflation end located on said lumen.

13. A multi-lumen balloon catheter and a tunnel balloon catheter according to claim 1, wherein a marker of any length and shape, which is made of a radio-opaque material, can be present in at least one of components such as the proximal end, a left ventricular outflow balloon, the aortic valve dilatation balloon, an ascending aortic dilatation balloon, and a tunnel.

14. A single lumen hourglass shaped balloon catheter according to claim 1, comprising an hourglass balloon that allows the 1st part of the TAVI valve to be opened and fixed to the sub-aortic intraventricular (LVOT) part when inflated, and allows the aortic valve structures to be opened and expanded and the 3rd part of the valve to be placed in the aorta that extends out over the coronary sinus by opening or expanding the 3rd part of the TAVI valve.

15. A single lumen hourglass shaped balloon catheter according to claim 14, wherein the hourglass balloon comprises a single piece in the form of an hourglass or 3 connected parts and that it can be inflated from a single point.

16. A single lumen hourglass shaped balloon catheter according to claim 14, wherein the marker, preferably the strip, of any length and shape made of radio-opaque material may be present in at least one of the components of the proximal end, hourglass balloon, an hourglass balloon pilot balloon, and the lumen leading to the proximal end number 1.

17. A tunnel hourglass shaped balloon catheter according to claim 1, wherein a one-way valve is available between the distal end and proximal end of a tunnel.

18. A tunnel hourglass shaped balloon catheter according to claim 1, wherein the one-way valve between the distal end and proximal end of a tunnel has two or three valves.

19. A multi lumen, single lumen or tunnel hourglass shaped balloon catheter according to claim 1, wherein they are made of any material suitable for the physiological environment and known to be usable under surgical or angiographic conditions.
